(19) United States
(12) Patent Application Publication
(10) Pub. No.: US 2016/0361449 A1
(43) Pub. Date: Dec. 15, 2016

(54) DEVICE AND METHOD FOR ELECTRON BEAM STERILIZATION

(71) Applicant: TETRA LAVAL HOLDINGS & FINANCE S.A., Pully (CH)

(72) Inventor: Alaa OMRANE, Lund (SE)

(73) Assignee: TETRA LAVAL HOLDINGS & FINANCE S.A., Pully (CH)

(21) Appl. No.: 15/121,541

(22) PCT Filed: Jan. 21, 2015
(86) PCT No.: PCT/EP2015/051076

§ 371 Date: Aug. 25, 2016

(30) Foreign Application Priority Data
Feb. 26, 2014 (SE) 1450224-9

(51) Int. Cl. A61L 2/08; B65B 55/08; H01J 33/04; A61L 2/28

(52) U.S. Cl. CPC A61L 2/087; A61L 2/28; B65B 55/08; H01J 33/04; A61L 2202/11; A61L 2202/14; A61L 2202/23

(57) ABSTRACT

Electron beam sterilizing device for sterilizing a packaging container by electron beam irradiation, the electron beam sterilizing device comprising: a housing enclosing an internal space and an electron beam generator arranged in the internal space for generating an electron beam, wherein the housing comprises an electron exit window, and wherein a temperature measurement device is provided for measuring a temperature in a travelling path of the electron beam.

The invention relates to a device and method for sterilization of a packaging container or a packaging material by electron beam irradiation.

Within for example the food industry, it is common practice to pack liquid and partly liquid food products in packaging containers manufactured from polymer materials such as for example PET, or from a packaging laminate comprising a core layer of paper or paperboard and one or more barrier layers of, for example, polymer material or aluminium foil. One packaging type is the “carton bottle” manufactured in a filling machine in that packaging blanks of the above-described packaging laminate are formed and sealed as a sleeve. Said sleeve is closed in one end in that a top of thermoplastic material is injection moulded directly on the sleeve end portion. The sheets of packaging laminate may be cut from a magazine reel of packaging laminate.

When the top is finished the packaging container is ready to be filled with product through the still open bottom, and then sealed and finally folded. Before the filling operation the packaging container undergoes treatment. If distribution and storage is to be made in chilled temperature the packaging container is disinfected, whereas if distribution and storage is to be made in ambient temperature, the packaging container needs to be sterilized. Sterilization is a term referring to any process that eliminates or kills microbial life, including transmissible agents such as for example fungi, bacteria, viruses and spores, which may be present on a surface of the packaging material or in a product. In the (food) packaging industry this is generally referred to as aseptic packaging, i. e. packaging sterilized products in sterilized packaging containers, i. e. keeping both the product and the packaging container free form living germs and microorganisms, so that the freshness of the product can be preserved without special cooling requirements, i. e. so that sterility can be maintained inside a packaging container although it is stored in ambient temperature. In this context the term “commercially sterile” is also commonly used and means in general the absence of microorganisms capable of growing in the food at normal non-refrigerated conditions at which the food is likely to be held during manufacture, distribution and storage. In this patent application the word “sterile” refers to a condition being at least commercially sterile.

One way to sterilize such packaging containers is to irradiate it by means of a low voltage electron beam emitted from an electron beam emitter. An example of linear irradiation by electron beam of ready-to-fill packaging containers is disclosed in the international patent publication WO 2005/002973. Other examples of irradiation of packaging containers, in these cases PET bottles, are described in for example WO 2011/011079 and EP 2 371 397, the latter describing a rotary system. In these systems emitters are used having a diameter small enough to be passed through a neck portion of the bottles. Low voltage electron beam can also be used for sterilizing a web of packaging material. The web is transported in between two opposed electron beam emitters having their electron exit windows facing the surfaces of the web. Web sterilization is for example described in the international publication WO2004/110868.

Electron beam sterilization devices, or emitters, are known which can be inserted into a packaging container for sterilizing the interior of the packaging container. Moreover, there are electron beam sterilization devices for sterilizing exterior surfaces of a packaging container.

In order to monitor correct operation of the electron beam emitters, and thereby being able to secure sterility, it is common practise to perform dosimetry tests. These tests are made regularly, generally daily, throughout the lifetime of the electron beam emitter. In general, dosimetry tests involve adding a dosimeter means, i.e. a patch reacting on radiation exposure, to a packaging container to measure if a correct absorbed dose is obtained during radiation. At the same time measurements of voltage and current are made in the electron beam emitter. The current over the filament is measured by comparing the current fed to the filament and current leaving the filament. In this way it is possible to determine the amount of electrons emitted from the filament. In addition, the voltage, i.e. the electric potential, between the electron exit window and the filament is measured. The measured value of voltage and current is then used as a set value during production of packaging containers. The current and voltage are continuously monitored during production, and as long as the value is not lower than the set value it is assumed that the packaging containers receive the correct dose.

It is an object of the present invention to provide an electron beam sterilization device and a method for sterilizing a packaging container or a web of packaging material allowing for a particularly reliable sterilization in that on-line measurement and control of the functionality of the electron beam sterilization device is improved.

The object is solved according to the invention with an electron beam sterilizing device according to claim 1 and a method for electron beam sterilization according to claim 12. Preferred embodiments are defined in the dependent claims and the following description, in particular taken together with the attached drawings.

The electron beam sterilizing device according to the invention is adapted to sterilize a packaging container or a packaging material web by electron beam irradiation and comprises a housing enclosing an internal space and an electron beam generator arranged in the internal space for generating an electron beam. The housing comprises an electron exit window. The electron beam sterilizing device further comprises a temperature measurement device for measuring, or detecting, at least one temperature in a travelling path of the electron beam. Said temperature is a measure of the electron energy delivered by the electron beam sterilizing device.

The method according to the invention is a method for sterilizing a packaging container or a packaging material web by electron beam irradiation. The method comprises the steps of: generating an electron beam in a housing, making the electron beam exiting the housing through an electron exit window, and measuring a temperature in a travelling path of the electron beam as a measure of the electron energy delivered by the electron beam. The inventive method can be carried out by the inventive electron beam sterilizing device.

In order to obtain a suitable sterilization, the irradiation dose of the electron beam sterilizing device has to be kept within a given range. If the delivered dose is too small the sterilization might not be adequate. If the dose is too high, the packaging material might be affected negatively. It is therefore generally desirable to determine the irradiation dose, in particular the intensity of the electron beam of an electron beam sterilizing device, when operating the electron beam sterilizing device.

A basic idea of the invention is to provide a device and a method for determining the irradiation dose or intensity of the electron beam (output dose/electron beam energy output) or the electron energy delivered by the electron beam of an electron beam sterilizing device. The irradiation dose or electron beam dose is determined through a measurement of a temperature in a travelling path of the electron beam. When the electron beam, i.e. the electrons emitted by the electron beam generator, hits an object, heat is generated and the respective object is heated. By correlating the temperature of the object to the output dose of the electron beam sterilizing device, the temperature measurement device can be used as a dose sensor of the electron beam sterilizing device. The temperature measurement takes place during the operation of the electron beam sterilizing device, i.e. while an electron beam is generated and the electron beam passes through the electron exit window. The electron beam path is particularly a path extending from the electron beam generator towards the packaging container or packaging material web to be sterilized, i.e. from the electron beam generator towards and through the electron exit window.

The temperature can generally be measured at any location in the travelling path (electron beam path), for example between the electron beam generator and the electron exit window. In one or more embodiments, however, the temperature is measured at the electron exit window and the measurement device is adapted to measure a temperature of or at the electron exit window. The temperature of the electron exit window depends on the electron energy or irradiation dose or beam intensity and, therefore, the irradiation dose can be determined by determining the temperature of the electron exit window. When the electron beam passes through the electron exit window, the energy of the electron beam is partly transferred to the electron exit window. The electron exit window therefore heats up when the electron beam sterilizing device is operated, and the temperature of the electron exit window is directly related to the irradiation dose, in particular the electron current, or electron energy, emitted by the electron beam sterilizing device (emitter). Measurement of the temperature of the electron exit window therefore provides a simple way of determining the irradiation dose. The temperature measurement device can, therefore, be used to monitor the irradiation dose.

In one or more embodiments of the invention, the temperature measurement device comprises a temperature sensor arranged at the electron exit window. The temperature sensor, which can for example be a thermocouple, preferably contacts the electron exit window and detects the temperature of at least one specified position of the electron exit window, in particular through direct contact. The temperature measurement device can comprise one or more temperature sensors. The temperature sensors may be located at different positions of the electron exit window in order to detect uniformity of the electron beam over the electron exit window.

In one or more embodiments of the invention the temperature measurement device is situated in the internal space of the housing, in the travelling path of the electrons, i.e. between the electron generator and the electron exit window. The temperature measurement device is at least partially covered by a medium adapted to create heat when impinged by electrons. The medium may be a ceramic material. A similar temperature device may be used outside the electron exit window, in positions where the temperature device is not in direct contact with the electron exit window, still being in the travelling path of the electrons.

In one embodiment of the invention the electron exit window comprises a support structure and a foil which is transmissible for electrons, and the temperature measurement device, in particular the temperature sensor, is adapted to measure a temperature of the support structure and/or the foil. This embodiment is based on the fact that the energy delivered by the electron beam sterilizing device, or emitter, is partially and linearly deposited on both the vacuum window (foil) and its support. Therefore, measuring the temperature on these items provides a simple way of determining the electron current itself. The support element may in particular be a grid-like structure, for example made of copper, having a plurality of free spaces or holes through which the electrons can pass. The foil is permeable for electrons and may be a metallic foil, such as for example a titanium foil. The thickness of the foil may be in the range of 4-12 micrometers. The foil covers the support structure. For a measurement of the temperature of the support structure and/or the foil, a sensing head of the measurement device, or temperature sensor, can in one embodiment directly contact the support structure and/or the foil.

In another embodiment of the invention the temperature measurement device, in particular the temperature sensor, is arranged on and/or within the support structure. The positioning of the temperature sensor on and/or within the support structure allows for a non-intrusive in situ dose/temperature measurement, in particular if the temperature sensor does not enter the free spaces of the support structure. The temperature sensor is preferably arranged such that it does not interfere or block the electron beam passing through the free spaces of the support structure. The temperature measurement device, in particular the temperature sensor, is preferably arranged on a surface of the supporting structure facing the internal space or on a surface opposite the internal space, so as to avoid blocking of the free spaces in the support structure. In another embodiment, the temperature sensor can also be embedded in the material of the support structure.

In order to obtain a particularly reliable and/or predictable relationship between the measured temperature and the irradiation dose, the temperature measurement device, in particular the temperature sensor, is arranged so as to measure a temperature in a central portion of the electron exit window. In particular, if the electron exit window is substantially symmetrical with regard to the central portion or point, the temperature at this point is a reliable measure of the electron current. In one embodiment of the invention, the electron exit window is circular.

In another embodiment of the invention the temperature measurement device is adapted to measure temperatures at at least two different positions of the electron exit window. Through temperature measurements at more than one position of the electron exit window, the reliability of the determination of the emitted electron irradiation dose can be enhanced. The temperature measurement device can comprise two or more separate temperature sensors for measuring temperatures at distinct positions at the electron exit window. If a field of temperature sensors is provided, it is also possible to determine a spatial radiation profile of the electron beam sterilization device.

In one embodiment of the invention, the temperature measurement device comprises a thermocouple. A thermocouple provides a sufficient accuracy as needed for the present application and a reliable and cost efficient way of measuring temperatures.

In another embodiment of the invention the temperature measurement device comprises a non-contacting temperature sensor for measuring a temperature of the electron exit window. For example, a thermometer for measuring thermal radiation can be used. The thermal radiation is used for determining the temperature of the electron exit window. A known device of this type is a pyrometer. The pyrometer can for example be placed outside the housing of the electron beam sterilizing device and such that it is passed by the electron beam sterilizing device between successive sterilizations of packaging containers. The electron beam sterilizing device is preferably kept operating between the successive sterilizations so that a stable temperature at the electron exit window is provided, i.e. the temperature of the electron exit window is substantially constant over time.

The reliability of the determination of the electron irradiation dose based on a temperature measurement of only a portion of the electron exit window also depends on the temperature profile at the electron exit window generated by the electrons passing through the electron exit window. The more predictable and the more evenly distributed the temperature profile, the more accurate the determination of the electron irradiation dose. It is therefore preferred according to the invention to use an electron beam generator having a predictable and/or homogeneous electron emitting profile.

In one embodiment of the invention, the electron beam sterilizing device comprises an electron irradiation dose calculator for calculating an electron irradiation dose, in particular an electron current, based on the measured temperature. The electron irradiation dose calculator uses calibration data defining a relationship between the measured temperature, in particular at a specific position of the electron exit window, and the electron irradiation dose emanating from the electron exit window.

In yet another embodiment of the invention the electron beam sterilizing device comprises a display for displaying the measured temperature and/or for displaying the electron irradiation dose determined based on the measured temperature. The display is operationally connected to the temperature sensor and/or the electron irradiation dose calculator.

Another embodiment is defined by an alarm device which is adapted to trigger an alarm signal when the measured temperature exceeds or falls below a predetermined threshold. The alarm device is operationally connected to the temperature measurement device and may be adapted to emit an acoustic and/or visual alarm signal. A maximum temperature at the electron exit window can be in the range of 300° C. to 400° C. Alternatively, a feedback signal is generated and sent to an irradiation control module if the temperature decreases below a chosen threshold temperature. An irradiation control module is a module controlling various processes with regard to the sterilization. A certain feedback signal may be arranged to generate appropriate measures to be taken.

In one or more embodiments of the method according to the invention an electron irradiation dose is determined based on the measured temperature. The determination of the electron irradiation dose preferably uses calibration data indicative of a relationship between the measured temperature, in particular at a specific position of the electron exit window, and the overall electron irradiation dose of the electron exit window. The measurement of the temperature takes place during operation of the electron beam sterilization device, i.e. while the electron beam passes through the electron exit window.

In the following, the invention will be further described in connection with the attached drawings, in which:

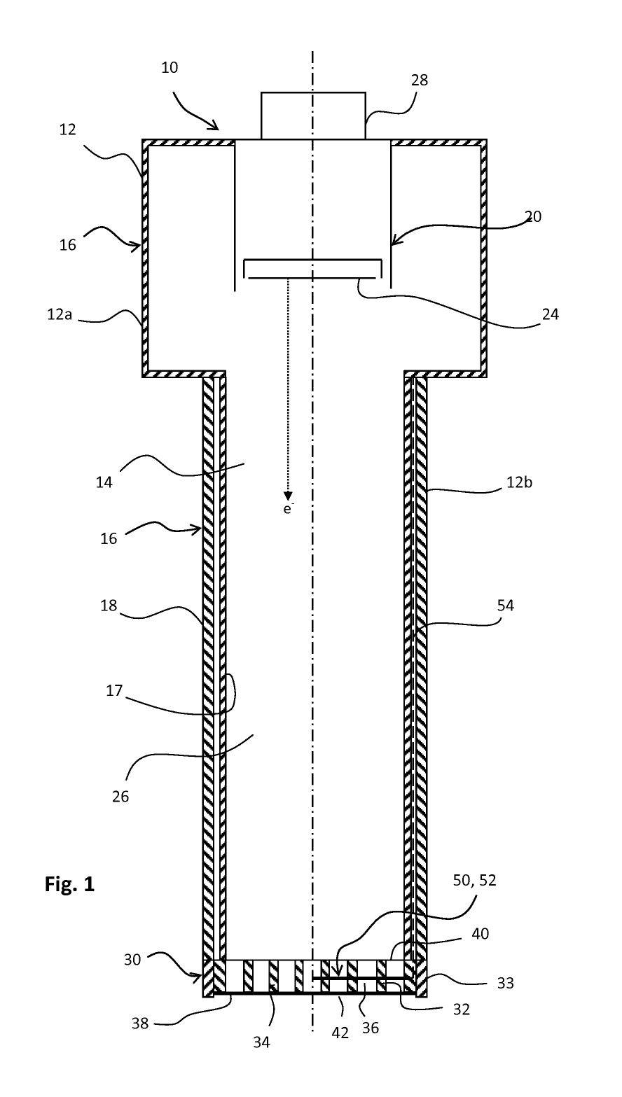

FIG. 1: shows an embodiment of an electron beam sterilization device according to the invention;

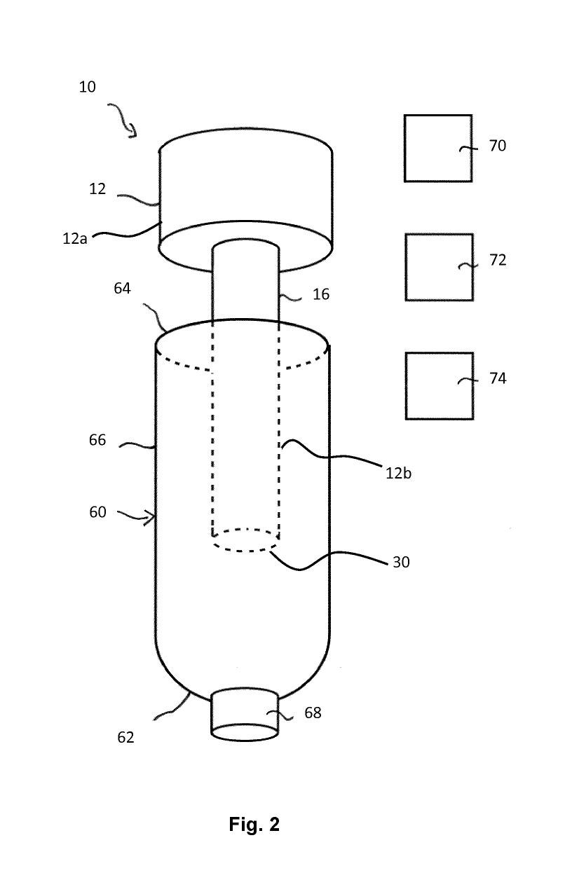

FIG. 2: shows a sterilization process of sterilizing an interior volume of a packaging container;

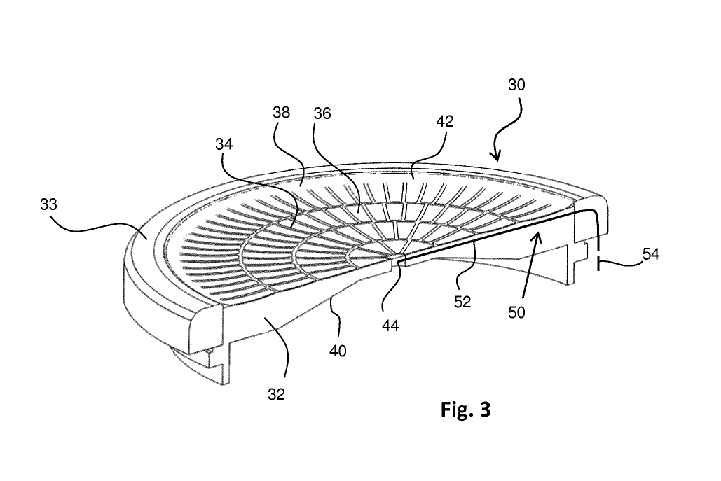

FIG. 3: shows another embodiment of an electron exit window according to the invention;

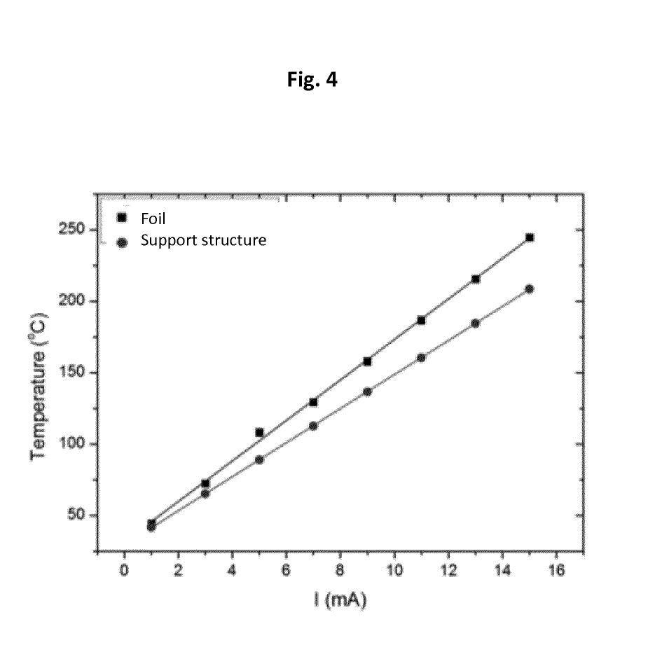

FIG. 4: shows a calibration curve for determining an electron current based on a measured temperature at an electron exit window.

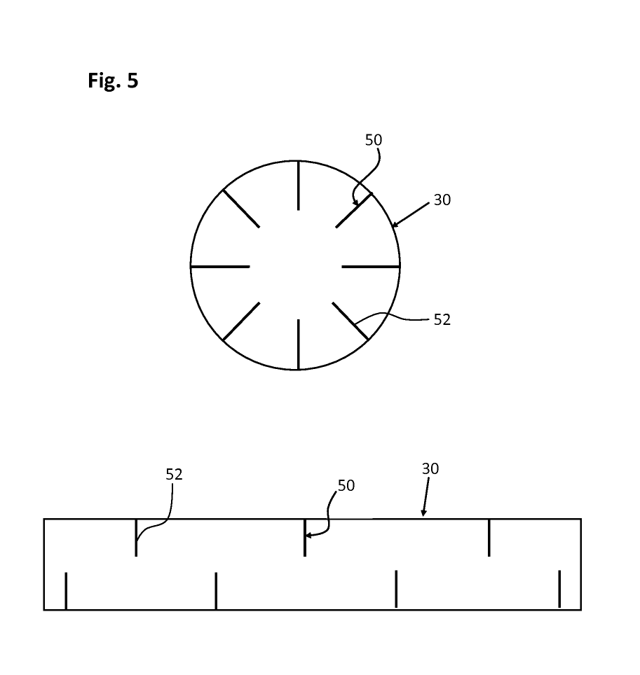

FIG. 5: shows a circular electron exit window and a rectangular electron exit window and the position of several temperature sensors.

Equal or corresponding elements are denominated with the same reference numerals in all figures. Features described in connection with different figures can be combined as long as technically possible.

FIG. 1 shows an electron beam sterilizing device 10 according to the invention for an interior sterilization of a packaging container 60, in particular a drug or food container.

The electron beam sterilizing device 10 comprises a housing 12 enclosing an internal space 14, in particular a vacuum space or vacuum chamber. An electron beam generator 20 is arranged in the housing 12, i.e. in the internal space 14. An electron exit window 30, which is permeable or transmissible for electrons, is arranged at an end surface of the housing 12. The housing 12 has a first part 12a provided with the electron generator 20, and a second part 12b provided with the electron exit window 30. The interior surface of a packaging container is sterilized by providing a relative movement between the electron beam sterilizing device 10 and the packaging container. Thus, the first part 12a has a larger diameter than the second part 12b, and the second part 12b has a diameter small enough to be inserted through an opening of the packaging container.

The electron beam generator 20 comprises a filament 24 which, when heated to an elevated temperature, in particular in the order of 2000° C., is caused to emit a cloud of electrons. The filament 24 can for example be made of tungsten. The electrons are accelerated in an electron acceleration zone 26 towards the electron exit window 30 by means of a voltage potential between the filament 24 (cathode) and the electron exit window 30 (anode). Due to the energy of the electrons, the electrons pass through the electron exit window 30 towards a target area. The target area is in this case the inside of a packaging container 60 to be sterilized, see FIG. 2. The acceleration of the electrons in the electron acceleration zone 26, FIG. 1, is achieved by a high-voltage field between the electron beam generator 20 and the electron exit window 30. The potential difference in the high-voltage field may be in the order of 75 kV to 150 kV. The filament 24 is coupled to a high voltage supply through a connector 28.

The housing 12 has a substantially longitudinal shape so as to be at least partially inserted into a packaging container 60 to be sterilized, as shown in FIG. 2. The electron beam sterilization device 10 can for example be inserted into the packaging container 60 through an open top or bottom end of the packaging container 60. In the embodiment shown in FIG. 2, the packaging container 60 has an upper side 62, a lower side 64 and a side wall 66 of a substantially tubular shape between the upper side 62 and the lower side 64. The electron beam sterilization device 10 is inserted into the packaging container 60 through the open lower side 64 of the packaging container 60. Alternatively, the electron beam sterilization device 10 may, however, also be inserted into the packaging container 60 through an opening in the upper side 62 of the packaging container 60. The upper side 62 can be spout, as shown in FIG. 2, which after filling can be closed by for instance a screw cap.

The electron exit window 30, best seen in FIG. 3, has a circular shape and comprises a grid-like support structure 32, for example made of aluminium or copper, and a metallic foil 38 which is transmissible for electrons. The metallic foil 38 is supported by the support structure 32 and can have a thickness in the order of 4 to 12 micrometers. The support structure 32, for example a grid or perforated plate, comprises a plurality of ribs 34 and a plurality of apertures 36 between the ribs 34. The ribs 34 extend at least partly in a radial direction of the electron exit window 30. The electron exit window 30 has an inner side 40 facing towards the internal space 14 or interior of the housing 12 and an outer side 42 facing, in use, towards the packaging container 60 to be sterilized.

For measuring a temperature at at least one position of the electron exit window 30 the electron beam sterilization device 10 comprises a temperature measurement device 50. The temperature measurement device 50 comprises a temperature sensor 52, for example a thermocouple, positioned at the support structure 32 of the electron exit window 30. In the FIG. 3 embodiment the temperature sensor 52 is arranged at or within one of the ribs 34 of the support structure 32, preferably a rib 34 extending in a radial direction. In order not to obstruct the apertures 36 of the support structure 32, the temperature sensor 52 can either be integrated into one of the ribs 34 or arranged on a surface of a rib 34, preferably on the outer side 42 or the inner side 40 of the electron exit window 30 or the support structure 32. Alternatively, and as is shown in FIG. 1, the temperature sensor 52 may extend partly through apertures 36 and ribs 34.

The temperature measurement device 50 further comprises a cable 54 for connecting the temperature sensor 52 to a signal converter, in particular for converting a voltage value to a temperature value. The cable 54 is routed along a side wall 16 of the housing 14, for example as shown in FIG. 1 where the cable 54 is illustrated as a dashed line. As can be seen in FIG. 1, the side wall 16 is composed of an inner wall 17 and an outer wall 18, and the cable 54 is routed through an interior space between the inner wall 17 and the outer wall 18. However, other arrangements are also possible.

FIG. 3 shows an embodiment in which the temperature sensor 52 of the temperature measurement device 50 extends at or within the support structure 32. It extends in a radial direction between a central position 44 of the electron exit window 30 and an outer support ring 33 of the support structure 32. The temperature sensor 52 is a thermocouple comprising two different metals as is generally known in the art. A diameter of the thermocouple can be in the range of 15 to 35 micrometres, in particular 20 to 30 micrometres. The measuring point of the thermocouple is arranged in a central area (central position 44) of the electron exit window 30 and contacts the support structure 32 and/or the foil 38. For generating a homogeneous temperature profile at the electron exit window 30, the ribs 34 of the support structure 32 may have a varying thickness between the central position 44 of the support structure 32 and a radially outer portion of the support structure 32.

FIG. 4 shows a calibration curve for calibrating the temperature measurement device 50. On the x-coordinate the electron current I is shown (mA) and on the y-coordinate the temperature is shown (° C.). There is a linear relationship between the temperature and the electron current (irradiation dose). It can be seen that the temperature of the foil is generally higher than the temperature of the support structure. By means of this calibration curve, the electron current and/or the irradiation dose can be determined based on a measured temperature.

An electron irradiation dose calculator 70 is provided for calculating an irradiation dose or beam intensity based on the measured temperature. The measured temperature and/or the irradiation dose can be displayed on a display 72. An alarm device 74 is arranged for providing an alarm signal if the measured temperature exceeds or falls below a predetermined threshold temperature.

The invention provides an effective way of determining the electron current of an electron beam emitter used for sterilizing packaging containers, in particular for use in the drug or food industry. The device is robust and can be manufactured at low costs. A thin thermocouple can be used and made sensitive to temperature variations as required. The measured temperature is directly related to the electron current so that the temperature is a direct measure of the irradiation dose. A simple calibration can be used for determining the irradiation dose. The temperature measurement device can therefore be used as a dose sensor and/or an alarm monitor.

An exemplary temperature measurement device 50 may comprise several temperature sensors 52 distributed over the electron exit window in order to be able to detect also the uniformity, and any irregularities of the uniformity, of the electron beam. The upper view of FIG. 5 shows in a very schematic way a circular electron exit window 30. A temperature measurement device 50 comprises eight temperature sensors 52 arranged in or at the electron exit window, and are able to measure the temperature at eight different positions of the window. The temperature sensors are arranged equally distributed along the perimeter of the electron exit window and pointing in a direction towards the centre of the electron exit window 30. They do not reach all the way into the centre, but measure a distance from the centre. This is one exemplary arrangement suitable for electron beams being ring-shaped, i.e. when intensity in the centre of the electron beam is less than near the perimeter of the electron beam. The lower view of FIG. 5 shows a rectangular electron exit window 30 and the position of several temperature sensors 52 substantially equally distributed over the length of the window.

It has been described that the temperature measurement device may be provided in an electron beam sterilizing device for sterilization of the interior surfaces of packaging containers. It should be understood that the temperature measurement device of the invention is also suitable for electron beam sterilizing devices used for sterilization of packaging material webs or exterior surfaces of packaging containers. Such sterilization is described in for example the international publication WO2004/110868. One example of an electron beam emitter suited for web sterilization and exterior surface sterilization of packaging containers is shown in the international publication WO2013/004565. One or more temperature measurement devices are fitted in the support structure. The support structure may be of the kind described in the international publication WO2011/096874.

### REFERENCE NUMERALS

10 electron beam sterilizing device
12 housing
14 internal space
16 side wall
17 inner wall
18 outer wall
20 electron beam generator
24 filament
26 electron acceleration zone
28 connector
30 electron exit window
32 support structure
33 support ring
34 rib
36 aperture
38 foil
40 inner side
42 outer side
44 central position
50 temperature measurement device
52 temperature sensor
54 cable
60 packaging container
62 upper side
64 lower side
66 side wall
68 filling hole
70 electron irradiation dose calculator
72 display
74 alarm device

1. Electron beam sterilizing device for sterilizing a packaging container or a packaging material web by electron beam irradiation, the electron beam sterilizing device comprising:
a housing enclosing an internal space;
an electron beam generator arranged in the internal space for generating an electron beam;
the housing comprising an electron exit window;
a temperature measurement device for measuring a temperature in a travelling path of the electron beam, said temperature being a measure of the electron energy delivered of the electron beam.

2. Electron beam sterilizing device according to claim 1, wherein the temperature measurement device is configured to measure a temperature of the electron exit window.

3. Electron beam sterilizing device according to claim 1, wherein the temperature measurement device comprises at least one temperature sensor arranged at the electron exit window.

4. Electron beam sterilizing device according to claim 1, wherein the electron exit window comprises a support structure and a foil which is transmissible for electrons, and the temperature measurement device is configured to measure a temperature of the support structure and/or the foil.

5. Electron beam sterilizing device according to claim 1, wherein the electron exit window comprises a support structure for supporting a foil which is transmissible for electrons, and the temperature measurement device is arranged on and/or within the support structure.

6. Electron beam sterilizing device according to claim 1, wherein the temperature measurement device is arranged so as to measure a temperature in a central portion of the electron exit window.

7. Electron beam sterilizing device according to claim 1, wherein the temperature measurement device comprises at least two temperature sensors located at different positions of the electron exit window, and that each temperature sensor is adapted to measure a temperature.

8. Electron beam sterilizing device according to claim 1, wherein the temperature measurement device comprises at least one thermocouple.

9. Electron beam sterilizing device according to claim 1, wherein the temperature measurement device comprises at least one non-contacting temperature sensor for measuring a temperature of the electron exit window.

10. Electron beam sterilizing device according to claim 1, wherein an electron irradiation dose calculator is provided for calculating an electron irradiation dose based on the measured temperature.

11. Electron beam sterilizing device according to claim 1, wherein an alarm device is provided which is configured adapted to trigger an alarm signal when the measured temperature exceeds or falls below a predetermined threshold.

12. Method for sterilizing a packaging container or a packaging material web by electron beam irradiation according to claim 1,
wherein the method comprises:
generating an electron beam in the housing,
the electron beam exiting the housing through the electron exit window, and
measuring a temperature in a travelling path of the electron beam as a measure of the electron energy delivered by the electron beam.

13. Electron beam sterilizing device for sterilizing a packaging container or a packaging material web by electron beam irradiation, the electron beam sterilizing device comprising:
a housing enclosing an internal space, the housing including opposite ends;
an electron beam generator positioned in the internal space of the housing at a location spaced from one of the ends of the housing;
the electron beam generator including a filament connectable to a voltage source and configured to be heated to an elevated temperature to emit a cloud of electrons in the internal space of the housing;
the one end of the housing including an exit window;
the internal space of the housing including an acceleration zone between the exit window and the electron beam generator in which the cloud of electrons is accelerated, by virtue of a voltage difference between the filament and the exit window during operation of the electron beam sterilizing device, so that an electron beam passes through the exit window and exits the housing; and
a temperature sensor located at the one end of the housing to measure temperature in a travelling path of the electron beam to provide a measure of electron energy delivered by the electron beam.

14. Electron beam sterilizing device according to claim 13, wherein the temperature sensor is positioned at the electron exit window.

15. Electron beam sterilizing device according to claim 13, wherein the electron exit window comprises an electron-transmissible foil supported on a support structure, the temperature sensor being positioned at the support structure.

16. Electron beam sterilizing device according to claim 13, wherein the temperature sensor is positioned to measure temperature in a central portion of the electron exit window.

17. Electron beam sterilizing device according to claim 13, wherein the temperature sensor is a first temperature sensor, and further comprising a plurality of other temperature sensors located at the one end of the housing to measure temperature and spaced apart from one another.

18. Electron beam sterilizing device according to claim 13, wherein the temperature sensor is a thermocouple.

19. Electron beam sterilizing device according to claim 13, wherein the temperature sensor is a non-contacting temperature sensor.
